(19) United States
(12) Patent Application Publication
(10) Pub. No.: US 2021/0171688 A1
(43) Pub. Date: Jun. 10, 2021

(54) BIOCOMPATIBLE COPOLYMER, CURABLE COMPOSITION, BIOCOMPATIBLE COATING LAYER AND BIOCOMPATIBLE DEVICE INCLUDING THE SAME

(71) Applicant: National Central University, Taoyuan City (TW)

(72) Inventors: Chun-Jen HUANG, Taoyuan City (TW); Jin-He KE, Hsinchu County (TW)

(21) Appl. No.: 17/080,299

(22) Filed: Oct. 26, 2020

Related U.S. Application Data
(60) Provisional application No. 62/944,538, filed on Dec. 6, 2019.

(51) Int. Cl. C08F 230/02; C08F 230/08; C08J 3/24; C09D 143/02; A61L 31/14; A61L 31/10

(52) U.S. Cl. CPC C08F 230/02; C08F 230/08; C08J 3/24; C08J 2343/02; A61L 31/14; A61L 31/10; C09D 143/02

(57) ABSTRACT

A biocompatible copolymer includes a phosphorylcholine-containing structural unit represented by formula (I), a siloxy-containing structural unit represented by formula (II), and a photoreactive structural unit represented by formula (III), wherein each of the substituents is given the definition as set forth in the Specification and Claims. A curable composition, a biocompatible coating layer, and a biocompatible device containing the biocompatible copolymer are also disclosed.

### CROSS-REFERENCE TO RELATED APPLICATION

This application claims priority of U.S. Provisional Application No. 62/944,538, filed on Dec. 6, 2019.

### FIELD

The present disclosure relates to a copolymer, and more particularly to a biocompatible copolymer. The present disclosure also relates to a curable composition including the biocompatible copolymer, a biocompatible coating layer including the curable composition, and a biocompatible device including the biocompatible coating layer.

### BACKGROUND

Taiwanese Invention Patent Publication No. TW 201634501 A discloses a copolymer including a phosphorylcholine-containing structural unit represented by formula (I),

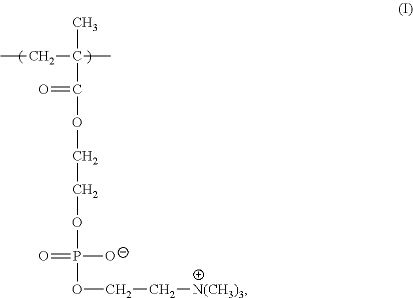

an alkyl-containing structural unit represented by formula (II),

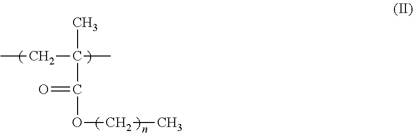

and a benzophenone-containing structural unit represented by formula (III),

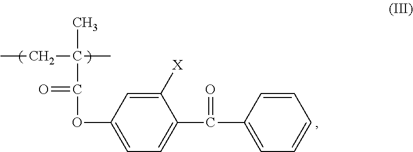

wherein, in formula (II), n is an integer ranging from 3 to 7, and in formula (III), X represents hydrogen or hydroxyl group.

The copolymer may be used as a surface treatment agent for treating a substrate surface of a medical device. After irradiation with light, the copolymer can form a cross-linked body on the substrate surface, thereby imparting the following advantageous characteristics to the resultant medical device: inhibition of protein adsorption, inhibition of cell adhesion, anticoagulation, and hydrophilic properties through the presence of the phosphorylcholine-containing structural unit represented by formula (I); enhancement of physical adhesion to the substrate surface through the presence of the alkyl-containing structural unit represented by formula (II); and capability of being chemically bonded to the substrate surface through the presence of the benzophenone-containing structural group represented by formula (III).

### SUMMARY

Therefore, an object of the present disclosure is to provide a biocompatible copolymer, a curable composition, a biocompatible coating layer, and a biocompatible device that can alleviate at least one of the drawbacks of the prior art.

According to one aspect of the present disclosure, the biocompatible copolymer includes a phosphorylcholine-containing structural unit represented by formula (I),

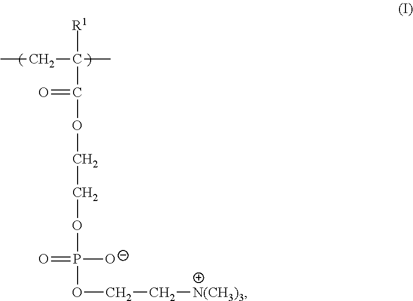

a siloxy-containing structural unit represented by formula (II),

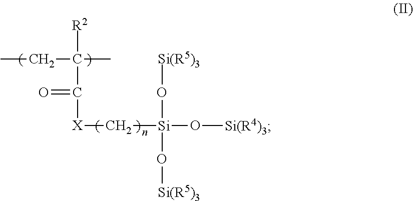

a photoreactive structural unit represented by formula (III),

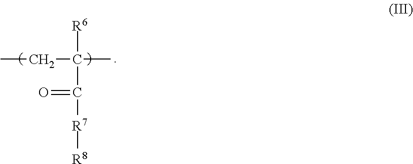

In formula (I), R¹ represents hydrogen or an alkyl group. In formula (II), R² represents hydrogen or an alkyl group; X represents N or O; n is an integer ranging from 1 to 14; R³, R⁴, and R⁵ independently represent an alkyl group or an aromatic group; each R³ is the same or different; each R⁴ is the same or different; and each R⁵ is the same or different. In formula (III), R⁶ represents hydrogen or an alkyl group; R⁷ represents N or O; and R⁸ represents a photoreactive group.

According to another aspect of the present disclosure, the curable composition includes the abovementioned biocompatible copolymer for treating a device.

According to yet another aspect of the present disclosure, the biocompatible coating layer is formed by curing the abovementioned curable composition. The biocompatible coating layer is adapted to be disposed on a substrate.

According to still yet another aspect of the present disclosure, the biocompatible device includes a substrate and the abovementioned biocompatible coating layer that is bonded to the substrate.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

This disclosure provides a biocompatible polymer which includes a phosphorylcholine-containing structural unit represented by formula (I), a siloxy-containing structural unit represented by formula (II), and a photoreactive structural unit represented by formula (III).

The phosphorylcholine-containing structural unit represented by formula (I) is shown as follows:

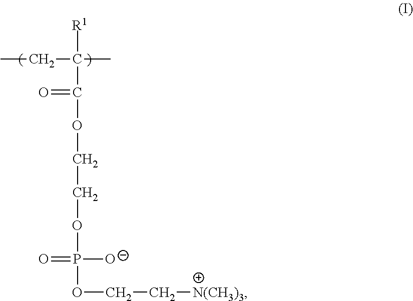

wherein, R¹ represents hydrogen or an alkyl group. An exemplary example of the alkyl group may include, but is not limited to, a methyl group.

In certain embodiments, the phosphorylcholine-containing structural unit represented by formula (I) is present in the biocompatible copolymer in a molar fraction ranging from 0.1 to 0.95.

The phosphorylcholine-containing structural unit represented by formula (I) may be derived from a phosphorylcholine-containing compound. Examples of the phosphorylcholine-containing compound may include, but are not limited to, 2-methacryloyloxyethyl phosphorylcholine (abbreviated as “MPC”), 2-acryloyloxyethyl phosphorylcholine, and a combination thereof.

The siloxy-containing structural unit represented by formula (II) is shown as follows:

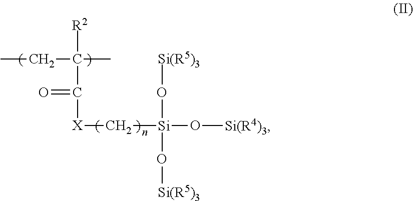

wherein, R² represents hydrogen or an alkyl group; X represents N or O; n is an integer ranging from 1 to 14; R³, R⁴, and R⁵ independently represent an alkyl group or an aromatic group; each R³ is the same or different; each R⁴ is the same or different; and each R⁵ is the same or different.

In R², the alkyl group may be, for example, a methyl group. In R³, R⁴, and R⁵, the alkyl group may be, for example, a methyl group or an ethyl group. In R³, R⁴, and R⁵, the aromatic group may be, for example, a phenyl group.

In certain embodiments, the siloxy-containing structural unit represented by formula (II) is present in the biocompatible copolymer in a molar fraction ranging from 0.01 to 0.6.

The siloxy-containing structural unit represented by formula (II) may be derived from a siloxy-containing compound which is represented by formula (II-1) shown as follows:

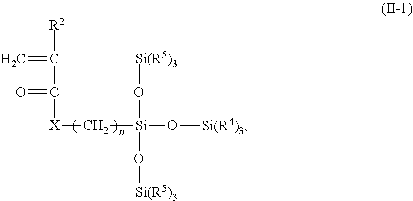

wherein, n, R², R³, R⁴, and R⁵ are the same as those defined above for formula (II), and thus are not described herein for the sake of brevity.

Examples of the siloxy-containing compound represented by formula (II-1) may include, but are not limited to, 3-[tris(trimethylsiloxy)silyl]propyl methacrylate (abbreviated as TSM), 3-[tris(trimethylsiloxy)silyl]propylacrylate, 3-[tris(trimethylsiloxy)silyl]propylacrylamide, 3-[tris(trimethylsiloxy)silyl]propyl methacrylamide, [tris(trimethylsiloxy)silyl]methyl methacrylate, [tris(trimethylsiloxy)silyl]methylacrylate, [(trimethylsiloxy)dimethylsilyl]methyl methacrylate, [(trimethylsiloxy)dimethylsilyl]methylacrylate, [(trimethylsiloxy)dimethylsilyl]methyl methacrylamide, and combinations thereof.

The photoreactive structural unit represented by formula (III) is shown as follows:

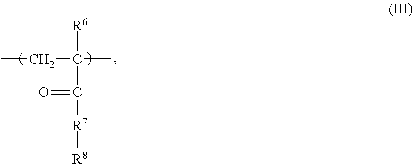

wherein, R⁶ represents hydrogen or an alkyl group; R⁷ represents N or O; and R⁸ represents a photoreactive group. An exemplary example of the alkyl group may include, but is not limited to, a methyl group.

In certain embodiments, the photoreactive structural unit represented by formula (III) is present in the biocompatible copolymer in a molar fraction ranging from 0.01 to 0.5.

Examples of the photoreactive group may include, but are not limited to,

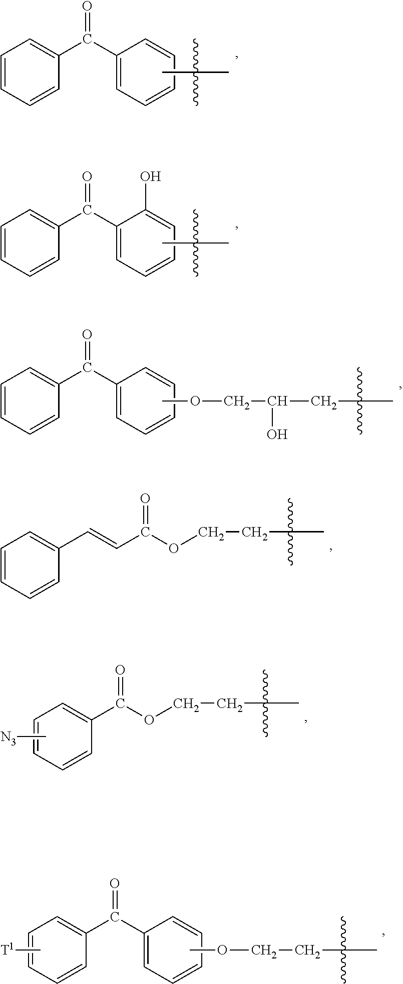

wherein, T¹ represents H, —OCH₃ or —CN.

The photoreactive structural unit represented by formula (III) may be derived from a photoreactive compound which is represented by formula (III-1) shown as follows:

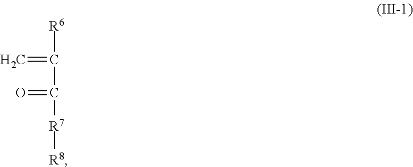

wherein, R⁶, R⁷, and R⁸ are the same as those defined above for formula (III), and thus are not described herein for the sake of brevity.

Examples of the photoreactive compound represented by formula (III-1) may include, but are not limited to,

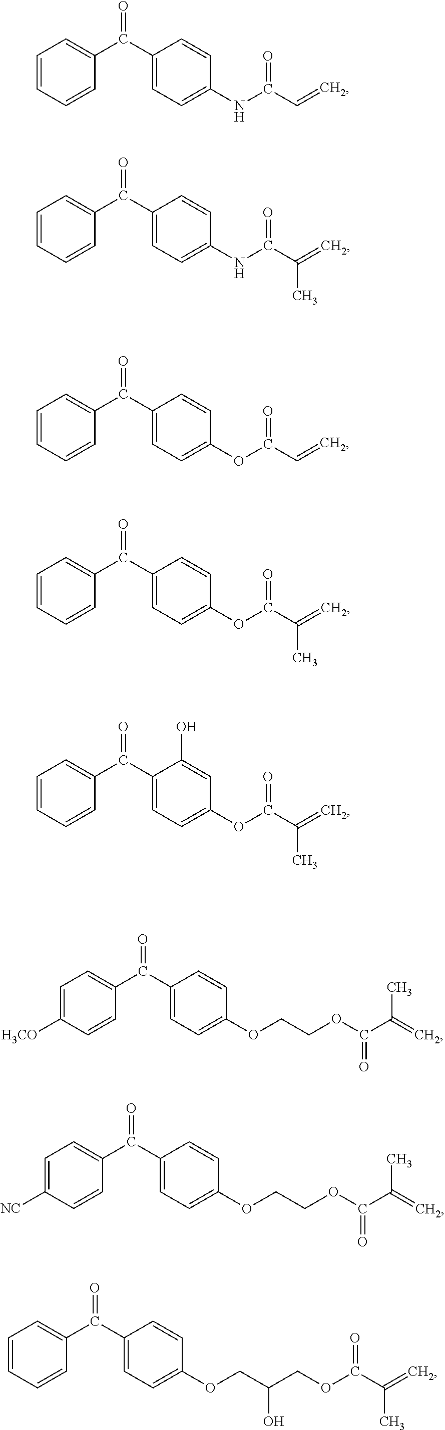

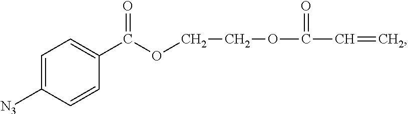 [SMILES: C=CC(=O)OCCOC(=O)c1ccc(N=[N+]=[N-])cc1]

and combinations thereof.

In certain embodiments, the biocompatible copolymer may have a weight average molecular weight that ranges from 10000 Da to 10000000 Da.

According to this disclosure, the biocompatible copolymer may be prepared by subjecting a composition that includes a phosphorylcholine-containing component, a siloxy-containing component and a photoreactive component to a polymerization reaction.

The phosphorylcholine-containing component may include at least one the abovementioned phosphorylcholine-containing compound. The siloxy-containing component may include at least one the abovementioned siloxy-containing compound represented by formula (II-1). The photoreactive component may include at least one the abovementioned photoreactive compound represented by formula (III-1). The composition may further include at least one solvent. Examples of the solvent may include, but are not limited to, water, methanol, ethanol, acetone, methyl ethyl ketone, tetrahydrofuran, and dimethylformamide.

The present disclosure also provides a curable composition for treating a device. The curable composition includes the abovementioned biocompatible polymer, and optionally, a solvent. An example of the solvent is ethanol, but is not limited thereto.

In certain embodiments, the curable composition further includes a reactive monomer, a cross-linking agent, and an initiator.

Examples of the reactive monomer may include, but are not limited to, 2-methacryloyloxyethyl phosphorylcholine, ethylene glycol acrylate, ethylene glycol methacrylate, ethylene glycol acrylamide, N-vinylpyrrolidone, and combinations thereof.

Examples of the cross-linking agent may include, but are not limited to, polyethylene glycol diacrylate (such as triethylene glycol diacrylate), polyethylene glycol dimethacrylate (such as diethylene glycol dimethacrylate), 1,6-hexanediol diacrylate, 1,9-nonanediol dimethacrylate, 1,4-butanediol dimethacrylate, 1,3-butanediol dimethacrylate, 1,10-decanediol dimethacrylate, 1,4-butanediol diacrylate, ethylene glycol diacrylate, and 1,5-pentanediol dimethacrylate.

The initiator may be a photo initiator compound or a thermal initiator compound. Examples of the initiator may include, but are not limited to, 4-benzoylphenyl acrylate, ethylpyruvate, ethyl-methyl-2-oxobutanoate, 4-4-dimethyldihydrofuran-2,3-dione, 4-benzoyl-4-methyldiphenyl sulphide, benzophenone, ethyl phenylglyoxylate, isopropylthioxanthone, 2-hydroxy-4′-(2-hydroxyethoxy)-2-methylpropiophenone (e.g., Irgacure® 2959, commercially available from Sigma-Aldrich), 4-[2-hydroxy-3-(N,N,N-trimethylammonium)propoxy]benzophenone chloride (abbreviated as Quantaacure BPQ}, ammonium persulfate-tetramethylethylenediamine, ammonium persulfate-sodium sulfite-Mohr's salt, and combinations thereof.

The present disclosure also provides a biocompatible coating layer adapted to be disposed on a substrate. The biocompatible coating layer is formed by curing the abovementioned curable composition.

The present disclosure also provides a biocompatible device including a substrate and the abovementioned biocompatible coating layer that is bonded to the substrate.

In certain embodiments, the substrate is made of a hydrogel material.

Examples of the biocompatible device may include, but are not limited to, a medical equipment (e.g., stent, catheter) and contact lens.

This disclosure will be further described by way of the following examples. However, it should be understood that the following examples are intended solely for the purpose of illustration and should not be construed as limiting the disclosure in practice.

### EXAMPLES

### Example 1 (E1)

First, a solvent component (including tetrahydrofuran and ethanol in a volume ratio of 1:1) was added to 2-methacryloyloxyethyl phosphorylcholine, 3-[tris(trimethylsiloxy)silyl]propyl methacrylate, and 4-benzoylphenyl acrylate in a round bottom flask, and then azobisisobutyronitrile was added under stirring so as to obtain a mixture. Next, the flask was deoxygenated using nitrogen for 30 minutes, and then capped with a nitrogen-filled balloon, followed by a polymerization reaction in an oil bath under heating at 60° C. for 16 hours. Thereafter, the resultant product was cooled and then subjected to a purification treatment by dialysis against methanol for two days. The dialysis product was concentrated under reduced pressure to remove methanol therefrom, and then dried under vacuum, thus obtaining a biocompatible copolymer of E1 as a whitish powder.

The biocompatible copolymer of E1 was then mixed with polyvinylpyrrolidone (PVP serving as an oxygen scavenger and a viscosity-adjusting agent), and ethanol to obtain a curable composition, which was used to treat a medical catheter made of a thermoplastic polyurethane material (Manufacturer: CUUMed Catheter Medical Co., Ltd., Taiwan).

To be specific, the medical catheter was cleaned using alcohol, and then immersed in an alcohol solution including alcohol and 1 wt % of benzophenone for 3 minutes, followed by drying in air for 30 minutes. After that, the sanitized catheter was immersed in the curable composition for 5 minutes, followed by subjected to irradiation using ultraviolet light having a wavelength of 254 nm for 15 minutes, so as to form a biocompatible coating layer on the catheter. Subsequently, the catheter having the biocompatible coating layer formed thereon was cleaned using deionized water and alcohol, thereby obtaining a biocompatible device of E1 for subsequent analysis.

### Examples 2 to 8 (E2 to E8)

The procedures for preparing a respective one of the biocompatible devices of E2 to E8 were similar to those of E1, except for differences in the molar percentage of the reacting components for forming the biocompatible copolymer, and in the amount of each component of the curable composition applied in E2 to E8, which are shown in Table 1 below.

### Control Example 1 (CE1)

The sanitized catheter, which is not treated with any curable composition, directly serves as a Control Example 1.

### Examples 9 to 17 (E9 to E17)

The procedures for preparing a respective one of the biocompatible devices of E9 to E17 were similar to those of E1, except that a medical device to be treated in E9 to E17 is a contact lens (Manufacturer: Yung Sheng Optical Co., Ltd., Taiwan). In addition, the molar percentage of the reacting components for forming the biocompatible copolymer, and the amount of each component of the curable composition applied in E9 to E17 are shown in Table 2 below.

### Control Example 2 (CE2)

The contact lens, which is cleaned with water and not treated with any curable composition, directly serves as a Control Example 2.

### Property Evaluation for Biocompatible Devices

### 1. Lubricity

First, each of the biocompatible devices of E1 to E17 was immersed in ethanol, and then taken out for rinse and dry. Next, each of the biocompatible devices of E1 to E17 was disposed on a medical-grade silicon film having a Shore hardness of 60 A which is laid on a bottom portion of a customized friction clamp device that is mounted on a tensile testing machine. After that, 1.1 L of deionized water was poured into a chamber of the friction clamp device such that each of the biocompatible devices was completely immersed in the deionized water for 1 minute. Thereafter, each of the biocompatible devices was tied to a 200 mg slider using a thin iron wire, and was subjected to a rubbing operation against the silicon film once at a speed of 150 mm/min to be displaced 130 mm, so as to determine a dynamic friction coefficient thereof after one-time rubbing operation, which serves as an indicator of lubricity. Results are shown in Tables 1 and 2 below, in which the symbols “O”, “Δ” and “X” respectively represent the determined dynamic friction coefficient of the biocompatible device being lower than 0.25, ranging from 0.25 to 1.0, and greater than 1.0.

### 2. Friction Resistance

The procedures for determining the friction resistance of each of the biocompatible devices of E1 to E17 are similar to those for determining lubricity as described above, except that the rubbing operation was repeated for 20 times, so as to determine a dynamic friction coefficient thereof after 20-times rubbing operations. The friction resistance for each of the biocompatible devices of E1 to E17, the catheter of CE1 and the contact lens of CE2 was calculated by subtracting the dynamic friction coefficient obtained after one-time rubbing operation from the dynamic friction coefficient obtained after 20 times rubbing operations. Results are shown in Tables 1 and 2 below, in which the symbols “O”, “Δ” and “X” respectively represent the thus obtained friction resistance being lower than 0.25, ranging from 0.25 to 1.0, and greater than 1.0.

### 3. Ethanol Wash Resistance

The biocompatible coating layer of each of the biocompatible devices of E1 to E17 was washed with 95% aqueous ethanol solution for three times, and then subjected to the measurement of friction resistance as mentioned above, so as to determine the ethanol wash resistance thereof. Results are shown in Tables 1 and 2 below, in which the symbols “O”, “Δ” and “X” respectively represent friction resistance being lower than <0.25, ranging from 0.25 to 1.0, and greater than 1.0.

### 4. Hydrophilicity

5 μL of deionized water was dropped on a surface of each of the biocompatible devices of E1 to E17, the catheter of CE1 and the contact lens of CE2 at three different positions, and hydrophilicity was determined by measuring an average value of water contact angle using a contact angle goniometer (Manufacturer: Surface Electro Optics, South Korea; Model No.: Phoenix Mini). Results are shown in Tables 1 and 2 below, in which the symbols “O”, “Δ” and “X” respectively represent the determined water contact angle being greater 45°, ranging from 45° to 75°, and greater than 750.

### 5. Protein Adsorption Resistance

Each of the biocompatible devices of E1 to E17, the catheter of CE1, and the contact lens of CE2 serving as a test sample was placed in a 2 mL microcentrifuge tube, and 2 mL of phosphate-buffered saline (PBS) was added thereto. Next, the microcentrifuge tube was placed in an incubator at a temperature of 37° C. and a speed of 80 rpm for 30 minutes. After removing the liquid from the microcentrifuge tube, 2 mL of bovine serum albumine (BSA) solution was added to be mixed with the test sample at a temperature of 37° C. and a speed of 80 rpm for 3 hours. The liquid was removed from the microcentrifuge tube, followed by washing with 2 mL of a PBST solution (i.e., PSB added with 0.05% Tween 80) three times at a speed of 80 rpm (5 minutes for each time), so as to remove the remaining BSA that was not adsorbed on the surface of the test sample. Thereafter, the test sample was incubated with 2 mL of a primary antibody solution (i.e., 5.5 μg/mL of an goat anti-BSA IgG antibody dissolved in PBST) at a temperature of 37° C. and a speed of 80 rpm for 1 hour, followed by washing with PBST three times at a speed of 80 rpm (5 minutes for each time). Then, the test sample was incubated with 2 mL of a secondary antibody solution (i.e., 5.5 μg/mL horseradish peroxidase (HRP)-conjugated anti-goat IgG secondary antibody dissolved in PBST) at a temperature of 37° C. and a speed of 80 rpm for 1 hour, followed by washing with PBST three times at a speed of 80 rpm (5 minutes for each time).

Subsequently, each of the test samples was transferred to a new microcentrifuge tube, and 1.5 mL of 3,3,5,5-tetramethylbenzidine (TMB) solution (serving as the substrate of HRP) was added to be mixed with the test sample, such that TMB was oxidized during the enzymatic degradation of H₂O₂ by HRP to form 3,3,5,5-tetramethylbenzidine diimine, which was exhibited as blue color. Thereafter, the resultant oxidized TMB solution of each of the test samples was transferred to a 24-well plate and left standing to allow the reaction to proceed at room temperature for 60 minutes. After that, the resultant reaction solution was added with 1 mL of aqueous sulfuric acid to terminate the reaction, and then subjected to light absorbance measurement at a wavelength of 450 nm (OD₄₅₀) using an ELISA reader (Manufacturer: BioTek Instruments; Model No.: Synergy™ HT). Relative protein adsorption amount on the test sample, i.e., protein adsorption resistance, can be calculated by substituting the light absorbance into the following formula:

A=(B/C)×100 (1)

where A=relative protein adsorption amount (%)
B=OD₄₅₀ of a respective one of the test samples
C=OD₄₅₀ of the test sample of the control example

Results are shown in Tables 1 and 2 below, in which for Table 1, the symbols “O”, “Δ” and “X” respectively represent the protein adsorption amount determined in the test sample being less than 20% of that of CE1, ranging from 20% to 50% of that of CE1, and greater than 50% of that of CE1, and for Table 2, the symbols “O”, “Δ” and “X” respectively represent the protein adsorption amount determined in the test sample is less than 20% of that of CE2, ranging from 20% to 50% of that of CE2, and greater than 50% of that of CE2.

### 6. Bacterial Adhesion Resistance

Escherichia coli cells and Staphylococcus epidermis cells were each inoculated into 10 mL LB media, and cultured at 37° C., 5% CO₂, and 150 rpm for 16 hours. After that, a respective one of Escherichia coli cell culture and Staphylococcus epidermis cell culture (i.e., bacterial cell culture) was subjected to the following experimental procedures.

The bacterial cell culture was subjected to centrifugation and the thus formed supernatant was removed, followed by resuspension in 10 mL PBS, so as to prepare a bacterial cell culture having a fixed concentration of OD₆₇₀=0.1.

Each of the biocompatible devices of E1 to E17, the catheter of CE1, and the contact lens of CE2 serving as test samples was placed in a 15 mL centrifuge tube, and 5 mL of the bacterial cell culture was added to completely cover the test sample. Next, each 15 mL centrifuge tube was incubated at 37° C., 5% CO₂, and 150 rpm for 3 hours. After removing the liquid from the 15 mL centrifuge tube, the test sample was washed with PBS, and then transferred to a new centrifuge tube containing 10 mL PBS. The new centrifuge tube was subjected to ultrasonic vibration for 15 minutes, so as to detach the adhered bacterial cells from the surface of the test sample. Thereafter, the detached bacterial cells was added with LB media to dilute 100 times, and then 10 μL of the thus diluted bacterial cells were plated on a LB agar plate, followed by culture at room temperature for 24 hours for observing growth of bacterial colonies. Bacterial adhesion resistance was determined by counting the bacterial colonies growing on the LB agar plate. Results are shown in Tables 1 and 2 below, in which for Table 1, the symbols “O”, “Δ” and “X” respectively represent the counted bacterial colonies for the test sample being less than 20% of that of CE1, ranging from 20% to 50% of that of CE1, and greater than 50% of that of CE1, and for Table 2, the symbols “O”, “Δ” and “X” respectively represent the counted bacterial colonies for the test sample being less than 20, of that of CE2, ranging from 20% to 50% of that of CE2, and greater than 50% of that of CE2.

### 7. Red Blood Cell Adhesion Resistance

Each of the biocompatible devices of E1 to E8 and the catheter of CE1 serving as test samples was placed in a whole blood solution, and then incubated for 2 hours at 37° C. After removing the whole blood solution, each of the test samples was washed twice using PBS to remove red blood cells not adhering to the surface thereof. After that, the red blood cells adhering to the surface of the test sample were fixed with ethanol and subjected to fluorescence staining, so as to observe distribution and number thereof using scanning electron microscopy or fluorescence microscopy.

Results are shown in Tables 1 and 2 below, in which the symbols “O”, “Δ” and “X” respectively represent the number of the adhered red blood cells on the test sample being less than 20% of that of CE1, ranging from 20 h to 50 h of that of CE1, and greater than 50% of that of CE1.

TABLE 1

| | | | | | | | | | | | Control |
|---|---|---|---|---|---|---|---|---|---|---|---|
| | | | Examples | | | | | | | | Example |
| Biocompatible device (catheter) | | | 1 | 2 | 3 | 4 | 5 | 6 | 7 | 8 | 1 |
| Curable | Biocompatible | Amount (wt %) | 2.5 | 5 | 2.5 | 1 | 1 | 1 | 1 | 1 | 0 |
| composition | copolymer | 2-methacryloyloxyethyl | 58.33 | | | | | | | | 0 |
| | | phosphorylcholine (mol %) | | | | | | | | | |
| | | 3-[tris(trimethylsiloxy) | 33.33 | | 25 | 33.33 | | | | | 0 |
| | | silyl]propyl methacrylate (mol %) | | | | | | | | | |
| | | 4-benzoylphenyl acrylate (mol %) | 8.34 | | 16.67 | 8.34 | | | | | 0 |
| | Cross-linking | Polyvinylpyrrolidone (wt %) | 2 | 2 | 2 | 0 | 1 | 2 | 2 | 2 | 0 |
| | agent | | | | | | | | | | |
| | Initiator | 4-benzoylphenyl acrylate (wt %) | 0 | 0 | 1 | 0 | 1 | 1 | 0 | 0.5 | 0 |
| | Solvent | Ethanol (wt %) | 95.5 | 93 | 94.5 | 99 | 97 | 96 | 97 | 96.5 | 0 |

| Property evaluation | Lubricity | ◯ | ◯ | ◯ | ◯ | Δ | ◯ | Δ | Δ | — |
|---|---|---|---|---|---|---|---|---|---|---|
| | Friction resistance | ◯ | ◯ | ◯ | Δ | Δ | Δ | X | X | — |
| | Ethanol wash resistance | ◯ | ◯ | ◯ | ◯ | Δ | ◯ | X | X | — |
| | Hydrophilicity | Δ | Δ | Δ | ◯ | ◯ | ◯ | Δ | ◯ | X |
| | Protein adsorption resistance | Δ | Δ | Δ | ◯ | Δ | ◯ | Δ | Δ | X |
| | Bacterial adhesion resistance | Δ | Δ | Δ | ◯ | Δ | ◯ | Δ | Δ | X |
| | Red blood cell adhesion resistance | Δ | Δ | Δ | ◯ | Δ | ◯ | Δ | Δ | X |

“—”: not performed

TABLE 2

| | | | | | | | | | | | | Control |
|---|---|---|---|---|---|---|---|---|---|---|---|---|
| | | | Examples | | | | | | | | | Example |
| Biocompatible device (contact lens) | | | 9 | 10 | 11 | 12 | 13 | 14 | 15 | 16 | 17 | 2 |
| Curable | Biocompatible | Amount (wt %) | 1 | 2.5 | 5 | 10 | 20 | 10 | 10 | 10 | 10 | 0 |
| composition | copolymer | 2-methacryloyloxyethyl | 70 | 70 | 70 | 70 | 70 | 50 | 60 | 80 | 90 | 0 |
| | | phosphorylcholine (mol %) | | | | | | | | | | |
| | | 3-[tris(trimethylsiloxy) | 25 | 25 | 25 | 25 | 25 | 45 | 35 | 15 | 5 | 0 |
| | | silyl]propyl methacrylate (mol %) | | | | | | | | | | |
| | | 4-benzoylphenyl acrylate (mol %) | 5 | 5 | 5 | 5 | 5 | 5 | 5 | 5 | 5 | 0 |
| | Solvent | Ethanol (wt %) | 99 | 97.5 | 95 | 90 | 80 | 90 | 90 | 90 | 90 | 0 |

| Property evaluation | Lubricity | ◯ | ◯ | ◯ | ◯ | ◯ | ◯ | ◯ | ◯ | ◯ | — |
|---|---|---|---|---|---|---|---|---|---|---|---|
| | Friction resistance | ◯ | ◯ | ◯ | ◯ | ◯ | ◯ | ◯ | Δ | X | — |
| | Ethanol wash resistance | ◯ | ◯ | ◯ | ◯ | ◯ | ◯ | ◯ | Δ | X | — |
| | Hydrophilicity | ◯ | ◯ | ◯ | ◯ | ◯ | Δ | ◯ | Δ | Δ | X |
| | Protein adsorption resistance | ◯ | ◯ | ◯ | ◯ | ◯ | ◯ | ◯ | Δ | Δ | X |
| | Bacterial adhesion resistance | ◯ | ◯ | ◯ | ◯ | ◯ | ◯ | ◯ | Δ | Δ | X |

“—”: not performed

In summary, the biocompatible copolymer of the present disclosure exhibits biocompatibility property with inclusion of the phosphorylcholine-containing structural unit, and is capable of being coated on a substrate of a biocompatible device through the siloxy-containing structural unit, and forming covalent bond with the substrate through the photoreactive structural unit after light irradiation, thereby forming a biocompatible coating layer firmly disposed on the substrate. In addition, the biocompatible copolymer of the present disclosure also has an excellent hydrophilicity and lubricity.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the present disclosure has been described in connection with what is considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A biocompatible copolymer, comprising:
a phosphorylcholine-containing structural unit represented by formula (I); 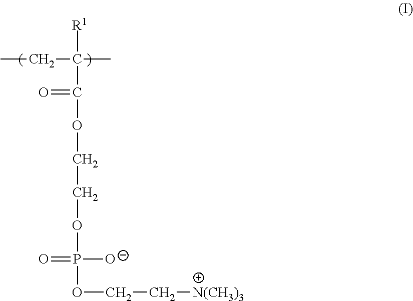
a siloxy-containing structural unit represented by formula (II); and 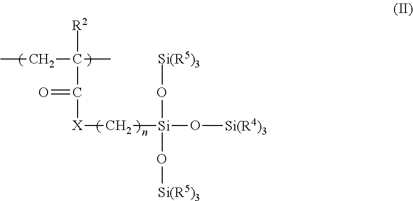
a photoreactive structural unit represented by formula (III), 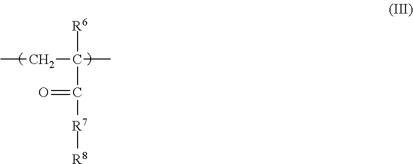
wherein in formula (I), R¹ represents hydrogen or an alkyl group,
wherein in formula (II), R² represents hydrogen or an alkyl group; X represents N or O; n is an integer ranging from 1 to 14; R³, R⁴, and R⁵ independently represent an alkyl group or an aromatic group; each R3 is the same or different; each R⁴ is the same or different; and each R⁵ is the same or different, and
wherein in formula (III), R⁶ represents hydrogen or an alkyl group; R⁷ represents N or O; and R⁸ represents a photoreactive group.

2. The biocompatible copolymer as claimed in claim 1, wherein said phosphorylcholine-containing structural unit represented by formula (I) is present in the biocompatible copolymer in a molar fraction ranging from 0.1 to 0.95.

3. The biocompatible copolymer as claimed in claim 1, wherein, said siloxy-containing structural unit represented by formula (II) is present in the biocompatible copolymer in a molar fraction ranging from 0.01 to 0.6.

4. The biocompatible copolymer as claimed in claim 1, wherein said photoreactive structural unit represented by formula (III) is present in the biocompatible copolymer in a molar fraction ranging from 0.01 to 0.5.

5. The biocompatible copolymer as claimed in claim 1, wherein said biocompatible copolymer has a weight average molecular weight that ranges from 10000 Da to 10000000 Da.

6. The biocompatible copolymer as claimed in claim 1, wherein in formula (III), said photoreactive structural unit is selected from the group consisting 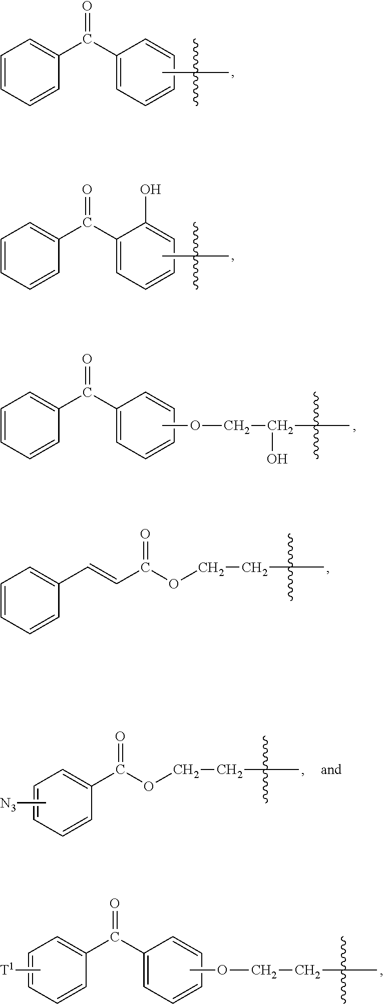
wherein T¹ represents H, —OCH₃ or —CN.

7. The biocompatible copolymer as claimed in claim 1, wherein said phosphorylcholine-containing structural unit represented by formula (I) is derived from a phosphorylcholine-containing compound selected from the group consisting of 2-methacryloyloxyethyl phosphorylcholine, 2-acryloyloxyethyl phosphorylcholine, and a combination thereof.

8. The biocompatible copolymer as claimed in claim 1, wherein said siloxy-containing structural unit represented by formula (II) is derived from a siloxy-containing compound selected from the group consisting of 3-[tris(trimethylsiloxy)silyl]propyl methacrylate, 3-[tris(trimethylsiloxy)silyl]propylacrylate, 3-[tris(trimethylsiloxy)silyl]propylacrylamide, 3-[tris(trimethylsiloxy)silyl]propyl methacrylamide, [tris(trimethylsiloxy)silyl]methyl methacrylate, [tris(trimethylsiloxy)silyl]methylacrylate, (trimethylsiloxy)dimethylsilyl]methyl methacrylate, [(trimethylsiloxy)dimethylsilyl]methylacrylate, [tris(trimethylsiloxy)silyl]methyl methacrylamide, and combinations thereof.

9. A curable composition for treating a device, comprising a biocompatible copolymer as claimed in claim 1.

10. A biocompatible coating layer adapted to be disposed on a substrate, said biocompatible coating layer being formed by curing a curable composition as claimed in claim 9.

11. A biocompatible device, comprising a substrate and a biocompatible coating layer as claimed in claim 10 that is bonded to said substrate.
